(19) United States
(12) Patent Application Publication
(10) Pub. No.: US 2022/0346857 A1
(43) Pub. Date: Nov. 3, 2022

(54) TISSUE ABLATION USING HIGH-FREQUENCY UNIPOLAR IRE

(71) Applicant: BIOSENSE WEBSTER (ISRAEL) LTD., Yokneam (IL)

(72) Inventors: Andres Claudio Altmann, Haifa (IL); Assaf Govari, Haifa (IL); Yuri Shamis, Haifa (IL)

(21) Appl. No.: 17/868,945

(22) Filed: Jul. 20, 2022

Related U.S. Application Data
(63) Continuation-in-part of application No. 16/701,989, filed on Dec. 3, 2019.
(60) Provisional application No. 63/241,782, filed on Sep. 8, 2021.

(51) Int. Cl. A61B 18/14

(52) U.S. Cl. CPC A61B 18/14; A61B 2018/00613

(57) ABSTRACT

A method for medical treatment includes providing a probe configured for insertion into a heart of a living subject and comprising at least one probe electrode configured to contact myocardial tissue in the heart. At least one body-surface electrode is configured to be fixed to skin of the living subject. Biphasic electrical pulses are applied between the at least one probe electrode and the at least one body-surface electrode with a peak-to-peak amplitude of at least 1 kV, a frequency of at least 500 kHz, and a current sufficient to cause irreversible electroporation of the myocardial tissue contacted by the at least one probe electrode.

### CROSS-REFERENCE TO RELATED APPLICATION

This application claims the benefit of U.S. Provisional Patent Application No. 63/241,782, filed Sep. 8, 2021, and is a continuation-in-part of U.S. patent application Ser. No. 16/701,989, filed Dec. 3, 2019.

### FIELD OF THE DISCLOSURE

The present disclosure relates generally to invasive medical equipment and procedures, and particularly to apparatus and methods for ablating tissue within the body.

### BACKGROUND

Irreversible electroporation (IRE) is a soft tissue ablation technique that applies short pulses of strong electrical fields to create permanent and hence lethal nanopores in the cell membrane, thus disrupting the cellular homeostasis (internal physical and chemical conditions). Cell death following IRE results from apoptosis (programmed cell death) and not necrosis (cell injury, which results in the destruction of a cell through the action of its own enzymes) as in other thermal and radiation-based ablation techniques. IRE is commonly used in tumor ablation in regions where precision and conservation of the extracellular matrix, blood flow and nerves are of importance.

IRE may also be applied in ablating tissue in the heart, for example as described in U.S. Patent Application Publication No. 2021/0161592, whose disclosure is incorporated herein by reference. As explained in this reference, IRE is a predominantly non-thermal process, which should cause an increase of the tissue temperature by, at most, a few degrees for a few milliseconds. It thus differs from RF (radio frequency) ablation, which raises the tissue temperature by between 20 and 70° C. and destroys cells through heating. IRE utilizes combinations of positive and negative pulses, which are applied, for example, between a bipolar pair of electrodes on a catheter. In order for the IRE pulses to generate the required nanopores in heart tissue, the field strength of the pulses must exceed a tissue-dependent threshold Eₜₕ, which for heart cells is approximately 500 V/cm. The applied voltages may reach up to 2000 V. The positive and negative pulses have pulse widths of 0.5-5 μs and a separation between the positive and negative pulses of 0.1-5 μs.

As another example, U.S. Patent Application Publication No. 2021/0177503, whose disclosure is incorporated herein by reference, describes a medical apparatus that includes a probe configured for insertion into a body of a patient and including a plurality of electrodes configured to contact tissue within the body. An electrical signal generator applies trains of positive and negative pulses having a voltage amplitude of at least 200 V and having a duration of each of the pulses less than 20 μs between at least one pair of the electrodes in contact with the tissue, thereby causing irreversible electroporation of the tissue between the at least one pair of the electrodes. One or more electrical sensors sense an energy dissipated between the at least one pair of the electrodes during the trains of the pulses. A controller controls electrical and temporal parameters of the trains of the pulses applied by the electrical signal generator, responsively to the one or more electrical sensors, so that the dissipated energy satisfies a predefined criterion.

In the above references, the pair of positive and negative pulses is referred to as a “bipolar pulse,” while application of the pulses between a pair of electrodes on a catheter is referred to as bipolar ablation (regardless of whether the pulses are positive or negative). To avoid confusion in the present description and in the claims, a pair of positive and negative pulses is referred to herein as a “biphasic pulse” or a biphasic pair of pulses.

### SUMMARY

Examples of the present disclosure that are described hereinbelow provide improved apparatus and methods for ablating tissue within the body.

### BRIEF DESCRIPTION OF THE DRAWINGS

The present disclosure will be more fully understood from the following detailed description of the examples thereof, taken together with the drawings in which:

FIG. 1 is a schematic pictorial illustration of a system used in an IRE ablation procedure, in accordance with examples of the disclosure;

FIG. 2A is a plot that schematically illustrates a biphasic IRE pulse, in accordance with an example of the disclosure; and

FIG. 2B is a plot that schematically illustrates a burst of biphasic IRE pulses, in accordance with an example of the disclosure.

### DETAILED DESCRIPTION OF EXAMPLES

IRE is typically performed by applying high-voltage pulses between pairs of electrodes that are relatively close to each other, to generate a high electrical field strength. In other words, IRE is typically a bipolar operation, and is implemented over a relatively small region. If bipolar IRE of this sort is to be applied to a large tissue region, such as for ablating the entire circumference of the ostium of a pulmonary vein (PV), for example, the ablation must be repeated multiple times over different parts of the region. This approach is time-consuming and may lead to inconsistent results, for instance due to cardiac motion that destabilizes catheter contact with the ostium.

For this reason, some IRE ablation procedures could advantageously be performed in a unipolar mode, between one electrode internal to the patient and another electrode on the patient's skin surface. In unipolar ablation, the IRE current spreads over a larger region around the internal electrode and thus creates a larger lesion at each location at which IRE is applied. When operating in IRE unipolar mode, however, the low equivalent capacitance of the patient gives rise to high impedance and can therefore reduce the efficiency of the procedure and make regulation of the electroporation difficult. Furthermore, because the current spreads over a larger region, the local current density may drop below the required level for effective IRE.

Examples of the present disclosure that are described herein overcome these limitations by applying unipolar IRE at voltage and frequency levels higher than were previously thought practicable. In the disclosed examples, an IRE signal generator applies biphasic electrical pulses between a probe electrode in the heart and a body-surface electrode on the subject's skin with a peak-to-peak amplitude of at least 1 kV, a frequency of at least 500 kHz, and a current sufficient to cause IRE of the myocardial tissue contacted by the probe electrode. In some examples, the peak-to-peak amplitude can be at least 2 kV, and the frequency can be 1 MHz or more. The high frequency of the electrical excitation counteracts the low capacitance of the patient. The high voltage drives high currents through the patient's body, typically at least 20 A, and possibly 30 A or more.

Thus, the unipolar IRE ablation proceeds efficiently and uniformly notwithstanding the wide spatial distribution of the current within the body. The biphasic pulses are typically applied in trains whose overall duration is less than 1 ms, so that the total energy delivered in each pulse train remains within safe limits.

Application of IRE at very high frequencies in the present examples is also advantageous in reducing muscle contractions due to the applied current. In bipolar IRE, in which the electrodes are close to one another, the currents are localized, and therefore lower frequencies can be used without causing muscle contractions. For unipolar IRE, the inventors have been found that higher frequencies, in excess of 500 kHz, are preferable. In order to reduce the current density in the muscles outside the heart, it is also desirable to use a large body-surface electrode, or even to use multiple body-surface electrodes, which are fixed to the skin at different, respective locations, so that the density of the current flowing through the muscles is further reduced.

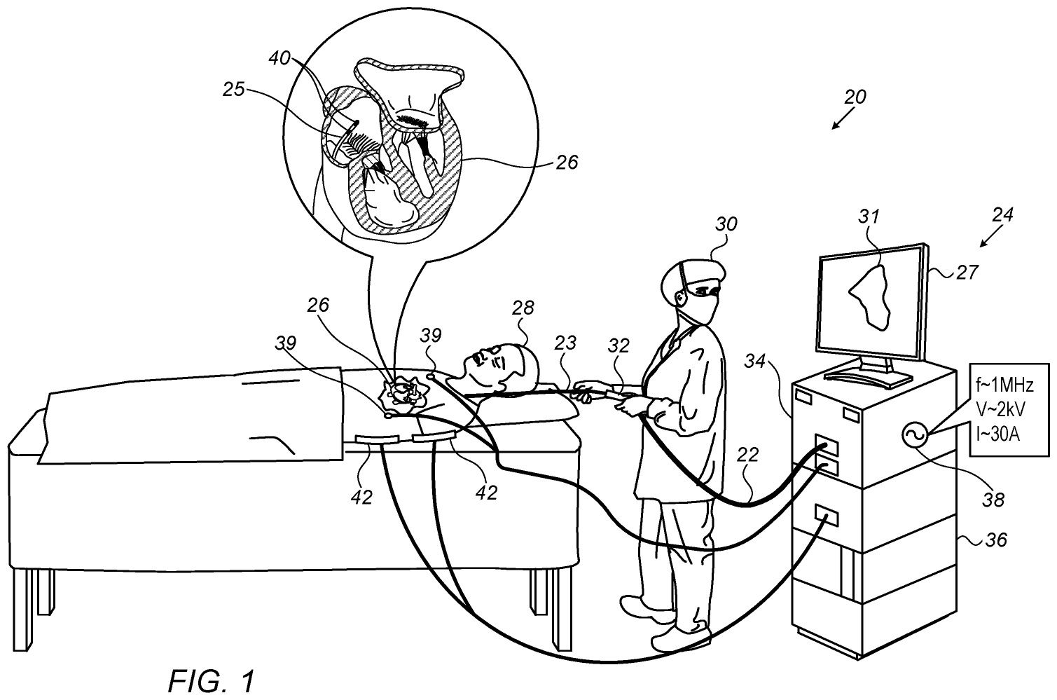

FIG. 1 is a schematic pictorial illustration of a system 20 used in an IRE ablation procedure, in accordance with an example of the disclosure. Elements of system 20 may be based on components of the CARTO® system, produced by Biosense Webster, Inc. (Irvine, Calif.).

A physician 30 navigates a catheter 22 via a sheath 23 through the vascular system of a patient 28 into a chamber of a heart 26 of the patient, and then deploys a distal end 25 of the catheter in the heart. Physician 30 steers distal end 25 using a manipulator 32 near the proximal end of catheter 22 so as to bring or more probe electrodes 40 on distal end 25 into contact with myocardial tissue at a site that is to be ablated. Although electrodes 40 are simply arrayed along the length of distal end 25 in FIG. 1, in alternative examples the catheter may comprise a distal structure, such as a basket, balloon, or lasso, along which the electrodes are disposed.

Catheter 22 is connected at its proximal end to a control console 24. A display 27 on console 24 may present a map 31 or other image of the heart chamber with an icon showing the location of distal end 25 in order to assist physician 30 in positioning electrodes 40 at the target location for the IRE ablation procedure. Console 24 may also receive, process, and make use of signals of other sorts, such as electrocardiogram (ECG) signals received from ECG electrodes 39 attached to patient 28.

Once distal end 25 is properly deployed and positioned in heart 26, physician 30 actuates an IRE module 34 in console 24 to apply sequences of IRE pulses to the electrodes on the basket assembly, under the control of a processor 36. The circuitry and other components of IRE module 34 are similar to those that are shown in FIGS. 1 and 5-9 of the above-mentioned U.S. Patent Application Publication 2021/0161592 and are described with reference thereto in the text of this publication. Specifically, IRE module 34 comprises an IRE signal generator 38 that is capable of generating biphasic pulses at frequencies up to at least 1 MHz, with peak-to-peak voltage of at least 2 kV, and power sufficient to drive a current of at least 30 A through electrodes 40. In the present example, the IRE pulses are applied in a unipolar mode between one or more of probe electrodes 40 and a separate common electrode, for example one or more conductive back patches 42, which are applied to the patient's skin. Alternatively or additionally, IRE pulses may be applied in a bipolar mode, between pairs of electrodes 40.

Processor 36 directs IRE module 34 to apply the pulses in accordance with a predefined protocol. Physician 30 may select the protocol to apply using controls on console 24. For example, the physician or an assistant may use a touch screen functionality of display 27 on console 24 to interact with processor 36. Alternatively or additionally, the physician or an assistant may operate the controls in order to select the pulse parameters and electrodes manually. In one example, processor 36 directs IRE signal generator 38 to synchronize the application of the IRE pulses with the heart cycle of patient 28, for example based on the ECG signals received from electrodes 39.

The system configuration that is shown in FIG. 1 is presented by way of example for conceptual clarity in understanding the operation of examples of the present disclosure. For the sake of simplicity, FIG. 1 shows only certain elements of system 20 that are related to these examples. The remaining elements of the system will be apparent to those skilled in the art, who will likewise understand that the principles of the present disclosure may be implemented in other medical therapeutic systems, using other components. All such alternative implementations are considered to be within the scope of the present disclosure.

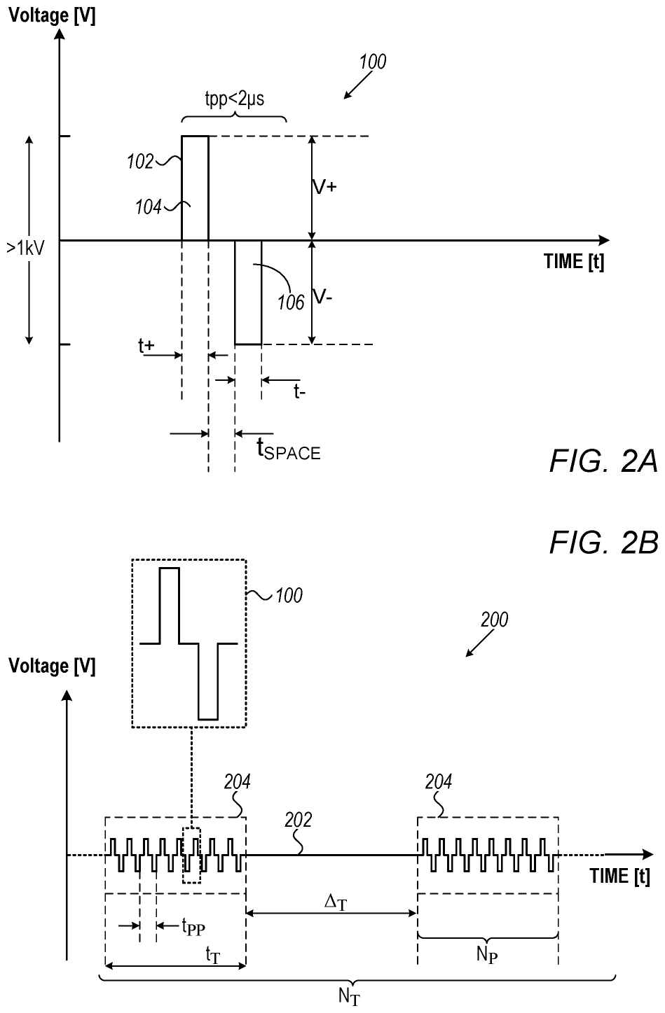

FIG. 2A is a schematic illustration of a biphasic IRE pulse 100, in accordance with an example of the disclosure. A curve 102 depicts the voltage V of pulse 100 as a function of time t in an IRE ablation procedure. The present example assumes that IRE signal generator 38 is configured as a voltage source, and the IRE signals are therefore described in terms of their voltages. Alternatively, IRE signal generator 38 may be configured as a current source, in which case the IRE pulses would be described in terms of their currents. Biphasic IRE pulse comprises a positive pulse 104 and a negative pulse 106, wherein the terms “positive” and “negative” refer to an arbitrarily chosen polarity of electrodes 40 and 42 between which the pulses are applied.

The amplitude of positive pulse 104 is labeled as V+, and the temporal width of the pulse is labeled as t+. Similarly, the amplitude of negative pulse 106 is labeled as V−, and the temporal width of the pulse is labeled as t−. The temporal width between positive pulse 104 and negative pulse 106 is labeled as t_{SPACE} The total peak-to-peak voltage of pulse 100 is Vₚₚ=V++V−, with Vₚₚ>1 kV for the unipolar IRE protocols that are described herein. The period of the biphasic pulses is tₚₚ=t++t−+2*t_{SPACE}, with tₚₚ<2 μs in order to achieve IRE pulse frequencies of 500 kHz or more.

FIG. 2B is a schematic illustration of a burst 200 of biphasic pulses 100, in accordance with an example of the disclosure. In the present example, the IRE signals are applied by IRE module 34 between electrodes 40 and 42 in one or more bursts 200, depicted by a curve 202. Burst 200 comprises N_{T} pulse trains 204, wherein each train comprises N_{P} biphasic pulses 100. The length of each pulse train 204 is labeled as t_{T}, and the interval between consecutive pulse trains is labeled as Δ_{T}, during which the signals are not applied. The length t_{T} is typically less than 1 ms, in order to avoid collateral damage to body tissues at the high voltage and power levels of pulses 100. In a typical IRE ablation procedure, t_{T}=100 μs, Δ_{T}=0.3-1000 ms, and N_{T} is between 1 and 100.

As one example, N_{T}=20 and Δ_{T}=5 ms, so that the total sequence of pulse trains extends over a duration of about 100 ms. The interval between pulse trains is helpful in avoiding excessive tissue heating. The inventors have found that sequences of pulse trains of this sort with an overall duration less than 250 ms give good results while minimizing collateral tissue damage.

### EXAMPLES

Example 1. A method for medical treatment, comprising providing a probe (22) configured for insertion into a heart (26) of a living subject (28) and comprising at least one probe electrode (40) configured to contact myocardial tissue in the heart; providing at least one body-surface electrode (42), which is configured to be fixed to skin of the living subject; and applying biphasic electrical pulses between the at least one probe electrode and the at least one body-surface electrode with a peak-to-peak amplitude of at least 1 kV, a frequency of at least 500 kHz, and a current sufficient to cause irreversible electroporation of the myocardial tissue contacted by the at least one probe electrode.

Example 2. The method according to example 1, wherein the peak-to-peak amplitude is at least 2 kV.

Example 3. The method according to example 1 or 2, wherein the frequency is at least 1 MHz.

Example 4. The method according to any of the preceding examples, wherein the current is at least 20 A.

Example 5. The method according to any of the preceding examples, wherein applying the biphasic electrical pulses comprises applying a train of the pulses having a train duration that is less than 1 ms.

Example 6. The method according to example 5, wherein applying the train of the pulses comprises applying a sequence of pulse trains with intervals between the pulse trains.

Example 7. The method according to example 6, wherein the sequence of the pulse trains has an overall duration less than 250 ms.

Example 8. The method according to any of the preceding examples, wherein providing the at least one body-surface electrode comprises providing multiple body-surface electrodes, which are fixed to the skin of the living subject at different, respective locations so that the current flows between the at least one probe electrode and the multiple body-surface electrodes.

Example 9. The method according to any of the preceding examples, wherein applying biphasic electrical pulses comprising synchronizing an application of the biphasic electrical pulses with a heart cycle of the living subject.

Example 10. Apparatus (20) for medical treatment, comprising a probe (22) configured for insertion into a heart (26) of a living subject (28) and comprising at least one probe electrode (40) configured to contact myocardial tissue in the heart; at least one body-surface electrode (42), which is configured to be fixed to skin of the living subject; and an IRE signal generator (34), which is configured to apply biphasic electrical pulses between the at least one probe electrode and the at least one body-surface electrode with a peak-to-peak amplitude of at least kV, a frequency of at least 500 kHz, and a current sufficient to cause irreversible electroporation of the myocardial tissue contacted by the at least one probe electrode.

Various features of the disclosure which are, for clarity, described in the contexts of separate examples may also be provided in combination in a single example. Conversely, various features of the disclosure which are, for brevity, described in the context of a single example may also be provided separately or in any suitable sub-combination.

It will be appreciated that the examples described above are cited by way of example, and that the present disclosure is not limited to what has been particularly shown and described hereinabove. Rather, the scope of the present disclosure includes both combinations and subcombinations of the various features described hereinabove, as well as variations and modifications thereof which would occur to persons skilled in the art upon reading the foregoing description and which are not disclosed in the prior art.

1. A method for medical treatment, comprising:
providing a probe configured for insertion into a heart of a living subject and comprising at least one probe electrode configured to contact myocardial tissue in the heart;
providing at least one body-surface electrode, which is configured to be fixed to skin of the living subject; and
applying biphasic electrical pulses between the at least one probe electrode and the at least one body-surface electrode with a peak-to-peak amplitude of at least 1 kV, a frequency of at least 500 kHz, and a current sufficient to cause irreversible electroporation of the myocardial tissue contacted by the at least one probe electrode.

2. The method according to claim 1, wherein the peak-to-peak amplitude is at least 2 kV.

3. The method according to claim 1, wherein the frequency is at least 1 MHz.

4. The method according to claim 1, wherein the current is at least 20 A.

5. The method according to claim 1, wherein applying the biphasic electrical pulses comprises applying a train of the pulses having a train duration that is less than 1 ms.

6. The method according to claim 5, wherein applying the train of the pulses comprises applying a sequence of pulse trains with intervals between the pulse trains.

7. The method according to claim 6, wherein the sequence of the pulse trains has an overall duration less than 250 ms.

8. The method according to claim 1, wherein providing the at least one body-surface electrode comprises providing multiple body-surface electrodes, which are fixed to the skin of the living subject at different, respective locations so that the current flows between the at least one probe electrode and the multiple body-surface electrodes.

9. The method according to claim 1, wherein applying biphasic electrical pulses comprising synchronizing an application of the biphasic electrical pulses with a heart cycle of the living subject.

10. Apparatus for medical treatment, comprising:
a probe configured for insertion into a heart of a living subject and comprising at least one probe electrode configured to contact myocardial tissue in the heart;
at least one body-surface electrode, which is configured to be fixed to skin of the living subject; and
an IRE signal generator, which is configured to apply biphasic electrical pulses between the at least one probe electrode and the at least one body-surface electrode with a peak-to-peak amplitude of at least 1 kV, a frequency of at least 500 kHz, and a current sufficient to cause irreversible electroporation of the myocardial tissue contacted by the at least one probe electrode.

11. The apparatus according to claim 10, wherein the peak-to-peak amplitude is at least 2 kV.

12. The apparatus according to claim 10, wherein the frequency is at least 1 MHz.

13. The apparatus according to claim 10, wherein the current is at least 20 A.

14. The apparatus according to claim 10, wherein the IRE signal generator is configured to apply a train of the biphasic electrical pulses having a train duration that is less than 1 ms.

15. The apparatus according to claim 14, wherein the IRE signal generator is configured to apply a sequence of pulse trains with intervals between the pulse trains.

16. The apparatus according to claim 15, wherein the sequence of the pulse trains has an overall duration less than 250 ms.

17. The apparatus according to claim 10, wherein the at least one body-surface electrode comprises multiple body-surface electrodes, which are configured to be fixed to the skin of the living subject at different, respective locations so that the current flows between the at least one probe electrode and the multiple body-surface electrodes.

18. The apparatus according to claim 10, wherein the IRE signal generator is configured to synchronize an application of the biphasic electrical pulses with a heart cycle of the living subject.
